(19) United States
(12) Patent Application Publication
(10) Pub. No.: US 2024/0188811 A1
(43) Pub. Date: Jun. 13, 2024

(54) ENDOSCOPE

(71) Applicant: FUJIFILM Corporation, Tokyo (JP)

(72) Inventors: Ukyo YAGYU, Kanagawa (JP); Naoki SUGIURA, Kanagawa (JP)

(73) Assignee: FUJIFILM Corporation, Tokyo (JP)

(21) Appl. No.: 18/516,865

(22) Filed: Nov. 21, 2023

(30) Foreign Application Priority Data
Dec. 7, 2022 (JP) 2022-195940

(51) Int. Cl. A61B 1/12; A61B 1/018

(52) U.S. Cl. CPC A61B 1/126; A61B 1/018

(57) ABSTRACT

An endoscope includes an insertion part to be inserted into a subject, a first surface disposed on a distal end surface of the insertion part, a forceps port disposed on the first surface, a second surface protruding from the first surface along an insertion direction of the insertion part, an observation window disposed on the second surface, a nozzle that is disposed on the first surface and jets a fluid from a jetting port toward the observation window, a third surface protruding from the first surface along the insertion direction of the insertion part, and a first illumination window that is disposed on the third surface. The third surface is located between the nozzle and the forceps port, and an end portion of the third surface in a jetting direction of the fluid is located further in the jetting direction than a formation position of the jetting port is.

### CROSS-REFERENCE TO RELATED APPLICATION

The present application claims priority under 35 U.S.C. § 119(a) to Japanese Patent Application No. 2022-195940 filed on Dec. 7, 2022, which is hereby expressly incorporated by reference, in its entirety, into the present application.

### BACKGROUND OF THE INVENTION

### 1. Field of the Invention

The present invention relates to an endoscope, and particularly relates to an endoscope for improving visibility of an observation window at a distal end portion of an insertion part.

### 2. Description of the Related Art

An observation window for introducing subject light from a site to be observed and an illumination window for emitting illumination light to the site to be observed with light are disposed on a distal end surface at a distal end portion of an insertion part of an endoscope. In addition, a fluid jetting nozzle (air/water supply nozzle) that jets cleaning water (water or the like) and a gas (air or the like) toward the observation window is disposed on the distal end surface.

In a case of cleaning the observation window, first, the cleaning water is jetted from a jetting port of the fluid jetting nozzle to remove the adherent substances adhering to the observation window, and then the gas is jetted from the jetting port to remove the cleaning water remaining on the observation window.

An endoscope disclosed in WO2014/030385A comprises an inclined portion at a peripheral edge portion of an observation window to make a second elevation angle along a second axis perpendicular to a first axis direction along a jetting direction larger than a first elevation angle in the first axis direction to thereby further improve cleaning and water draining properties of the observation window.

### SUMMARY OF THE INVENTION

Even in a case where a gas is jetted from a fluid jetting nozzle to remove cleaning water from an entire surface of an observation window, the cleaning water may remain in a region adjacent to the nozzle. In a case where the cleaning water is drawn out during an air supply operation of the nozzle and moves onto the observation window, there is a concern that the cleaning water is reflected in an observation image, which causes a decrease in visibility of the observation window.

The present invention has been made in consideration of such circumstances, and an object of the present invention is to provide an endoscope capable of suppressing the reflection of the cleaning water and improving the visibility of the observation window.

An endoscope of a first aspect comprises an insertion part that is inserted into a subject, a first surface that is disposed on a distal end surface of the insertion part, a forceps port that is disposed on the first surface, a second surface that protrudes from the first surface along an insertion direction of the insertion part, an observation window that is disposed on the second surface, a nozzle that is disposed on the first surface and that jets a fluid from a jetting port toward the observation window, a third surface that protrudes from the first surface along the insertion direction of the insertion part, and a first illumination window that is disposed on the third surface, in which the third surface is located between the nozzle and the forceps port, and an end portion of the third surface in a jetting direction of the fluid is located further in the jetting direction than a formation position of the jetting port is.

In an endoscope of a second aspect, the third surface comprises a protruding region including the end portion and extending in the jetting direction between a formation position of the first illumination window and the nozzle.

In an endoscope of a third aspect, the third surface is tapered toward the end portion in the jetting direction along the nozzle from an outer periphery of the distal end surface.

In an endoscope of a fourth aspect, a ridge line of the third surface on a side of the nozzle extends along the nozzle.

In an endoscope of a fifth aspect, a second illumination window is disposed on the second surface on a side opposite to the nozzle with the observation window interposed therebetween.

In an endoscope of a sixth aspect, a narrow portion formed by the second surface and the third surface is provided.

In an endoscope of a seventh aspect, a distance between a tail of the second surface and a tail of the third surface in the narrow portion is shorter than a distance between the jetting port and the tail of the second surface.

According to the present invention, it is possible to suppress the reflection of the cleaning water and to improve the visibility of the observation window.

### BRIEF DESCRIPTION OF THE DRAWINGS

FIG. 1 is a configuration view showing an endoscope according to an embodiment of the present invention.

FIG. 2 is a view for explaining a structure of a distal end portion of a first embodiment.

FIG. 3 is a perspective view of the distal end portion of the first embodiment.

FIG. 4 is a view for explaining other operations and effects of the first embodiment.

FIG. 5 is a view for explaining a structure of a modification example of the first embodiment.

FIGS. 6A and 6B are views for explaining a structure of a distal end portion of a second embodiment.

FIG. 7 is a view for explaining a structure of Modification Example 1 of the second embodiment.

FIG. 8 is a view for explaining a structure of Modification Example 2 of the second embodiment.

FIGS. 9A and 9B are views for explaining a structure of Modification Example 3 of the second embodiment.

FIG. 10 is a view showing a schematic configuration of a distal end portion and a bendable portion.

FIGS. 11A to 11D are views for explaining a first method of bonding a close contact spring to a PTFE tube.

FIGS. 12A to 12D are views for explaining a second method of bonding the close contact spring to the PTFE tube.

FIG. 13 is a view showing an example of a high-frequency treatment tool.

FIG. 14 is a schematic cross-sectional view of the distal end portion.

### DESCRIPTION OF THE PREFERRED EMBODIMENTS

Hereinafter, preferred embodiments of an endoscope according to the present invention will be described in detail with reference to the accompanying drawings.

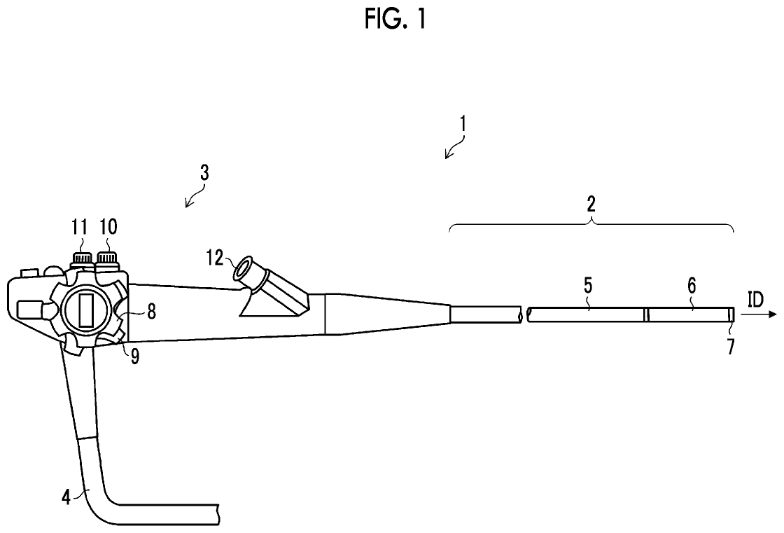

FIG. 1 is a configuration view showing an endoscope 1 according to the embodiment of the present invention. The endoscope 1 in FIG. 1 comprises an insertion part 2 that is inserted into a subject, an operating part 3 that is connected to a proximal end of the insertion part 2 and that is used for gripping and operating the endoscope 1, and a universal cord 4 that connects the endoscope 1 to a system constituent device such as a light source device and a processor device (not shown). The endoscope 1 of the present embodiment is an upper endoscope for observing an esophagus, a stomach, or the like.

The insertion part 2 has a distal end, a proximal end, and a longitudinal axis, and is composed of a soft portion 5, a bendable portion 6, and a distal end portion 7 that are consecutively provided in order from the proximal end toward the distal end. The soft portion 5 is flexible and is bendable in any direction along an insertion path of the insertion part 2. The operating part 3 is provided with angle knobs 8 and 9, a treatment tool inlet port 12, an air/water supply button 10, and a suction button 11.

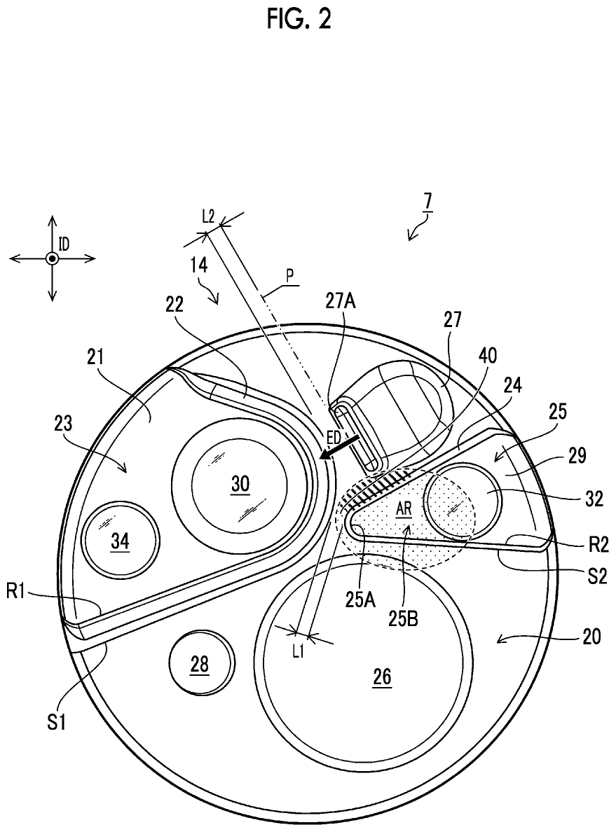

The bendable portion 6 is bent in each of up-down and left-right directions by the operation of each of the angle knobs 8 and 9. A treatment tool, such as a forceps, is inserted from the treatment tool inlet port 12 and is led out from a forceps port 26 (see FIG. 2) provided in the distal end portion 7. In addition, the distal end portion 7 is provided with an observation window 30 (see FIG. 2) for imaging a site to be observed in a body, and a first illumination window 32 and a second illumination window 34 (see FIG. 2) for irradiating the site to be observed with illumination light.

The insertion part 2 is inserted into a subject along an insertion direction ID indicated by an arrow, and the angle knobs 8 and 9 of the operating part 3 are rotationally operated to bend the bendable portion 6 of the insertion part 2 in the up-down and left-right directions. Accordingly, the distal end portion 7 of the insertion part 2 can be directed to a desired direction in the body, and an observation image can be acquired by the observation window 30 provided in the distal end portion 7.

### First Embodiment

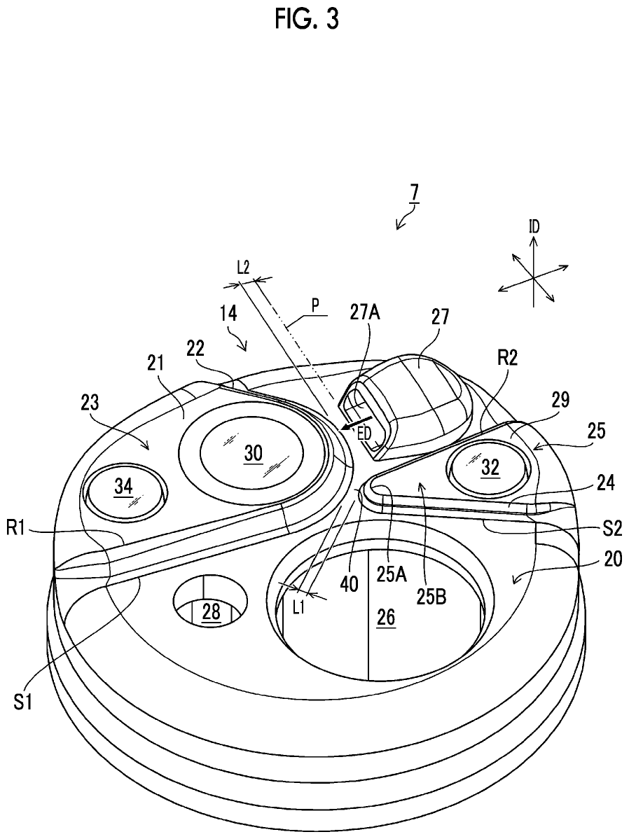

FIG. 2 is a view showing a distal end portion of a first embodiment as viewed from the insertion direction ID, and FIG. 3 is a perspective view of the distal end portion of the first embodiment. As shown in FIG. 2, the distal end portion 7 has a distal end surface 14 disposed on a distal end side in the insertion direction ID. The distal end surface 14 is formed in a circular shape as viewed from the insertion direction ID. The distal end surface 14 of the first embodiment comprises a first surface 20, a second surface 23 that protrudes from the first surface 20 in the insertion direction ID, and a third surface 25 that protrudes from the first surface 20 in the insertion direction ID. The forceps port 26 and a nozzle 27 are disposed on the first surface 20. The observation window 30 and the second illumination window 34 are disposed on the second surface 23. The first illumination window 32 is disposed on the third surface 25. Hereinafter, a direction (that is, the insertion direction ID) perpendicular to the distal end surface 14 is defined as a height direction (the same applies to a second embodiment which will be described later). In the first embodiment, the second surface 23 and the third surface 25 are surfaces higher than the first surface 20.

The observation window 30 is a constituent element of an observation unit that acquires an image of the site to be observed in order to observe the inside of the subject and introduces subject light from the site to be observed to an optical system (a lens group or the like) and to an imaging element which are other constituent elements of the observation unit. The image captured by the observation unit is sent as an observation image to a processor device connected by the universal cord 4.

The first illumination window 32 and the second illumination window 34 are constituent elements of an illumination unit mounted on the distal end portion 7, and irradiate the site to be observed with illumination light emitted from a light emitting unit which is another constituent element of the illumination unit. The illumination light emitted from the light emitting unit propagates through a light guide that is inserted through the inside of the endoscope 1 from a light source device connected by the universal cord 4.

The first surface 20 is composed of a flat surface perpendicular to the insertion direction ID. The first surface 20 is provided with the forceps port 26, the nozzle 27 for fluid jetting, and a front water jetting (WJ) nozzle 28.

The forceps port 26 communicates with the treatment tool inlet port 12 via a forceps tube, and the treatment tool inserted from the treatment tool inlet port 12 is led out from the forceps port 26. In addition, the forceps port 26 is connected to a suction pump (negative pressure source) via the forceps tube. By operating the suction button 11, cleaning water and adherent substances (blood or the like in the subject) are sucked from the forceps port 26.

The nozzle 27 comprises a jetting port 27A for jetting a fluid (a liquid or a gas), and the jetting port 27A is directed toward the observation window 30. The jetting port 27A of the nozzle 27 jets the fluid to a surface of the observation window 30 and to a peripheral portion thereof in a jetting direction ED indicated by an arrow and dispels and removes the cleaning water remaining on the observation window 30 located on a side in the jetting direction ED.

The front water jetting nozzle 28 jets a liquid such as cleaning water or a chemical liquid toward the site to be observed. The front water jetting nozzle 28 communicates with a water jet pipe line (not shown) disposed in the insertion part 2, the operating part 3, and the universal cord 4, and directly sprays the liquid sent from a liquid supply device, to the site to be observed. The front water jetting nozzle 28 is disposed at a position adjacent to the forceps port 26 on the first surface 20.

As viewed from the insertion direction ID, the second surface 23 extends from an outer periphery of the distal end surface 14 toward the nozzle 27 and has a substantially triangular shape. The second surface 23 is separated from the forceps port 26 and the nozzle 27.

The second surface 23 includes a flat surface 21 that is formed perpendicular to the insertion direction ID, and a stepped portion 22 which is a portion connecting the flat surface 21 and the first surface 20. The flat surface 21 is composed of a surface parallel to the first surface 20. The stepped portion 22 is composed of an inclined surface that rises from the first surface 20 toward the flat surface 21.

A ridge line R1, which is an intersection portion between the flat surface 21 and the stepped portion 22 of the second surface 23, is composed of a curved portion that follows the shape of the observation window 30 and two substantially straight portions from the curved portion toward the outer periphery of the distal end surface 14. An outer periphery of the second surface 23 has a curved shape that follows the shape of the outer periphery of the distal end surface 14. A tail S1, which is an intersection portion between the first surface 20 and the stepped portion 22, is composed of a curved portion that follows the shape of the observation window 30 and two substantially straight portions from the curved portion toward the outer periphery of the distal end surface 14.

In the flat surface 21 of the second surface 23, the observation window 30 is disposed on a side of the nozzle 27, and the second illumination window 34 is disposed on a side opposite to the nozzle 27 with the observation window 30 interposed therebetween.

As viewed from the insertion direction ID, the third surface 25 extends from the outer periphery of the distal end surface 14 toward the second surface 23 and is located between the forceps port 26 and the nozzle 27. An outer periphery of the third surface 25 has a curved shape that follows the shape of the outer periphery of the distal end surface 14.

The third surface 25 includes a flat surface 29 formed perpendicular to the insertion direction ID, and a stepped portion 24 which is a portion connecting the flat surface 29 and the first surface 20. The flat surface 29 is composed of a surface parallel to the first surface 20. The stepped portion 24 is composed of an inclined surface that rises from the first surface 20 toward the third surface 25.

FIG. 3 shows a height relationship among the first surface 20, the second surface 23, the third surface 25, and the nozzle 27. In a case where the first surface 20 is used as a reference position in the height direction (insertion direction ID), a maximum height position of each portion is higher in the order of the third surface 25, the second surface 23, and the nozzle 27. The first illumination window 32 is disposed on the third surface 25. From the viewpoint of improving visibility by broadening a light distribution area over a wide range toward the insertion direction ID, it is desirable that the third surface 25 has a lower height than the second surface 23. The height relationship between the third surface 25 and the second surface 23 is not necessarily limited in carrying out the present invention, and, for example, the third surface 25 and the second surface 23 may have the same height.

A structure of the distal end surface 14 of the first embodiment which is a feature part of the present invention will be described in more detail.

First, in a case of cleaning the observation window 30, the nozzle 27 jets cleaning water from the jetting port 27A toward the observation window 30 to remove the cleaning water and the adherent substance (blood, body fluid, or the like) adhering to the observation window 30. Next, the nozzle 27 jets a gas from the jetting port 27A to remove the cleaning water remaining on the observation window 30 or on an adjacent region thereof.

However, there may be a case where the cleaning water is not dispelled with the fluid from the nozzle 27 and the cleaning water remains on the first surface 20. In this state, in a case where the nozzle 27 performs an air supply operation, there is a problem in that a phenomenon in which a part of the cleaning water is drawn out (so-called stringing phenomenon) occurs, the cleaning water moves to the observation window 30, and the cleaning water is reflected in the observation image acquired from the observation window 30, which causes a decrease in the visibility of the observation window 30.

Accordingly, as a result of thoroughly investigating a countermeasure for suppressing a phenomenon in which the cleaning water is moved to the observation window 30 on the first surface 20, the inventors focused on a fact that the cleaning water remaining in an adjacent region AR adjacent to the nozzle 27 (in particular, a region including a nozzle side region of the nozzle 27 shown by diagonal lines on the forceps port 26 side) moves to and is reflected in the observation image and found that it is effective to relatively reduce the nozzle side region in particular in the adjacent region AR that is present on the first surface 20 by providing the third surface 25 having a height higher than the first surface 20. Hereinafter, the shape of the third surface 25 will be described in detail.

As shown in FIGS. 2 and 3, the third surface 25 has a shape of a substantially isosceles triangle as viewed from the insertion direction ID. A ridge line R2 which is an intersection portion between the flat surface 29 and the stepped portion 24 of the third surface 25 includes two straight portions. The two straight portions of the ridge line R2 extend from the outer periphery of the distal end surface 14 toward the side in the jetting direction ED to be close to each other and are connected to each other to form an end portion 25A of the third surface 25 in the jetting direction ED. The straight portion of the ridge line R2 on the nozzle 27 side extends along the nozzle 27 from the outer periphery of the distal end surface 14. Here, the expression “extends along the nozzle 27” means that the ridge line R2 may be roughly formed along an outer shape (outer shape on the ridge line R2 side) of the nozzle 27 at a position adjacent to the nozzle 27 and does not necessarily have to follow the outer shape of the nozzle 27. A tail S2, which is an intersection portion between the first surface 20 and the stepped portion 24, includes two straight portions.

In the first embodiment, in a case where a formation position of the jetting port 27A in the jetting direction ED is P, the end portion 25A of the third surface 25 is located further in the jetting direction ED than the formation position P of the jetting port 27A is. In other words, in a case of being viewed from a direction perpendicular to the distal end surface 14 and perpendicular to the jetting direction ED, the end portion 25A of the third surface 25 is located between the jetting port 27A and the observation window 30. At least a partial region of the end portion 25A of the third surface 25 only needs to be located further in the jetting direction ED than the formation position P of the jetting port 27A is. That is, the entire region of the end portion 25A of the third surface 25 does not necessarily have to be located further in the jetting direction ED than the formation position P of the jetting port 27A is.

Here, a plan shape in a case where the third surface 25 is viewed from the insertion direction ID will be described. The third surface 25 is a substantially isosceles triangle and is formed in a shape tapered toward the jetting direction ED (toward the end portion 25A). That is, the third surface 25 comprises a protruding region 25B (region including the end portion 25A) extending in the jetting direction ED. By having such a tapered shape, even in a case where a region (adjacent region AR) between the forceps port 26 and the nozzle 27 is narrow, at least a part of the end portion 25A of the third surface 25 (that is, the protruding region 25B including the end portion 25A) can be disposed further in the jetting direction ED than the formation position P of the jetting port 27A is. In the first embodiment, the ridge line R2 of the third surface 25 includes two straight portions, and the ridge line R2 does not follow the shape of the first illumination window 32.

As described above, in the first embodiment, since the third surface 25 having the above-described configuration is disposed on the distal end surface 14, a size occupied by the adjacent region AR (particularly, the nozzle side region) of the nozzle 27 on the first surface 20 can be made relatively small, and a residual amount of the cleaning water on the distal end surface 14 (adjacent region AR) can be suppressed as a whole. As a result, in a case where the air supply operation of the nozzle 27 is performed, the stringing phenomenon of the cleaning water is unlikely to occur, the reflection of the cleaning water on the observation image can be suppressed, and the visibility of the observation window 30 can be improved.

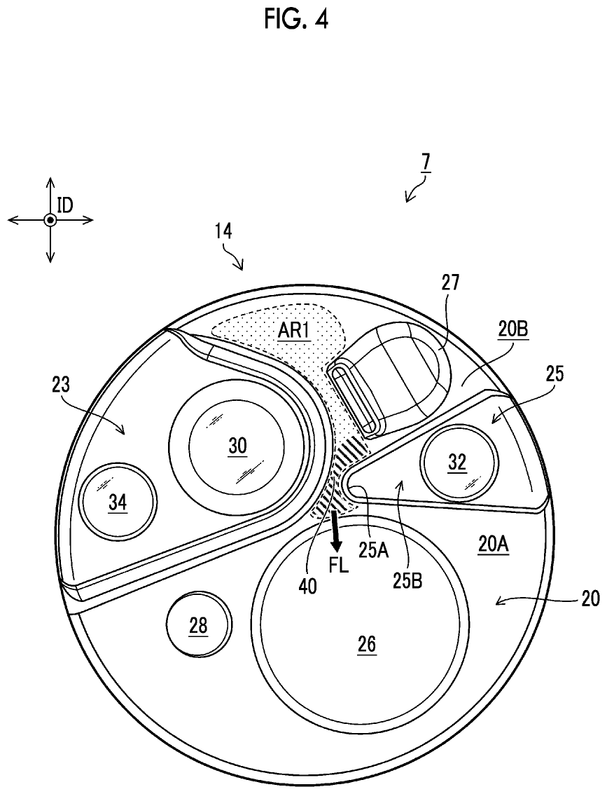

Next, a narrow portion 40 provided between the second surface 23 and the third surface 25 will be described. As shown in FIGS. 2 and 3, the second surface 23 and the third surface 25 are disposed to be spaced apart from each other, and the narrow portion 40 is formed between the second surface 23 and the third surface 25. In a case where the distal end surface 14 is viewed from the insertion direction ID, the first surface 20 is formed of two surfaces with the narrow portion 40 interposed therebetween, one surface of the two surfaces is a forceps port disposition surface 20A on which the forceps port 26 is disposed, and the other surface is a nozzle disposition surface 20B on which the nozzle 27 is disposed (see FIG. 4).

As shown in FIGS. 2 and 3, a width of the narrow portion 40 (the shortest distance between the second surface 23 and the third surface 25) is configured to be smaller than the shortest distance between the jetting port 27A and the second surface 23. Specifically, a distance L1 between the tail S1 of the second surface 23 and the tail S2 of the third surface 25 in the narrow portion 40 is configured to be shorter than a distance L2 between the jetting port 27A and the tail S1 of the second surface 23.

According to the configuration in which the narrow portion 40 is provided between the second surface 23 and the third surface 25 as described above, even in a case where an adherent substance having high viscosity (blood, body fluid, or the like) remains in a jetting port peripheral region AR1 in the nozzle disposition surface 20B of the first surface 20, the adherent substance is allowed to accumulate in, particularly, a residual region indicated by diagonal lines of the jetting port peripheral region AR1 so that a negative pressure generated in the forceps port 26 by a suction operation can be efficiently applied to the adherent substance in the residual region. As a result, the adherent substance can be sucked into the forceps port 26 along an arrow direction FL. In the embodiment of the present invention, the width of the narrow portion 40 does not necessarily have to be smaller than the shortest distance between the jetting port 27A and the second surface 23. However, from the viewpoint of efficiently applying the negative pressure to the adherent substance remaining in the jetting port peripheral region AR1 to be sucked into the forceps port 26, it is desirable that the width of the narrow portion 40 is configured to be smaller than the shortest distance between the jetting port 27A and the second surface 23. Although the suction of the adherent substance has been described, even in a case where the cleaning water remains, it is possible to suck the cleaning water into the forceps port 26 through the suction operation in the same manner as the adherent substance.

As described above, according to the first embodiment, the third surface 25 higher than the first surface 20 is provided between the forceps port 26 and the nozzle 27, and furthermore, the end portion 25A of the third surface 25 in the jetting direction ED is disposed further in the jetting direction ED than the formation position of the jetting port 27A of the nozzle 27 is. Therefore, the adjacent region AR (particularly, the nozzle side region) on the first surface 20 can be made relatively small, and a residual amount of the cleaning water on the first surface 20 can be suppressed as a whole. As a result, in a case where the air supply operation of the nozzle 27 is performed, the stringing phenomenon of the cleaning water is unlikely to occur, the reflection of the cleaning water on the observation image can be suppressed, and the visibility of the observation window 30 can be improved.

Further, according to the first embodiment, due to the narrow portion 40 provided between the second surface 23 and the third surface 25, the negative pressure generated in the forceps port 26 can be efficiently applied to the adherent substance remaining in the jetting port peripheral region AR1 (particularly, the residual region) on the first surface 20, and the adherent substance can be sucked by the forceps port 26. Further, even in a case where the cleaning water remains in the jetting port peripheral region AR1 of the first surface 20, it is possible to suck the cleaning water into the forceps port 26 through the suction operation in the same manner as the adherent substance.

### MODIFICATION EXAMPLE

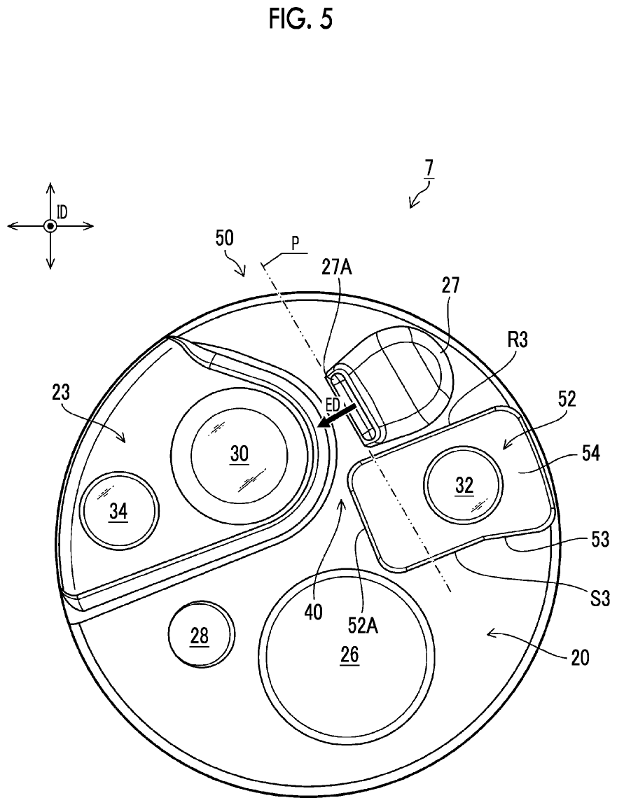

Next, a modification example of the distal end surface of the first embodiment will be described. FIG. 5 is a view for explaining the modification example. In FIG. 5, the same parts as those in the above-described first embodiment are designated by the same references, and the description thereof will not be repeated.

In a distal end surface 50 shown in FIG. 5, as viewed from the insertion direction ID, a shape of a third surface 52 is different from the shape of the third surface 25 of the above-described embodiment, and the third surface 52 in the modification example has a substantially rectangular shape. The third surface 52 includes a flat surface 54 formed perpendicular to the insertion direction ID, and a stepped portion 53 which is a portion connecting the flat surface 54 and the first surface 20. The first illumination window 32 is disposed on the flat surface 54.

A ridge line R3 between the flat surface 54 and the stepped portion 53 of the third surface 52 includes three straight portions and is composed of two parallel straight portions extending from an outer periphery of the distal end surface 50 toward the jetting direction ED and one straight portion that connects the two straight portions on a side further in the jetting direction ED than the formation position P of the jetting port 27A is. The one straight portion that connects the two straight portions constitutes an end portion 52A.

A tail S3 between the first surface 20 and the stepped portion 53 includes three straight portions as in the ridge line R3 and is composed of two parallel straight portions extending from the outer periphery of the distal end surface 50 toward the jetting direction ED and one straight portion that connects the two straight portions on a side further in the jetting direction ED than the formation position P of the jetting port 27A is.

Even in a case where a space between the forceps port 26 and the nozzle 27 is wider than that of the above-described first embodiment, by making the third surface 52 in a substantially rectangular shape, the adjacent region AR (particularly, the nozzle side region) (both not shown) on the first surface 20 can be more effectively made relatively small. As a result, since the cleaning water can be prevented from remaining on the first surface 20, movement of the cleaning water to the observation window 30 can be suppressed.

### Second Embodiment

Next, a second embodiment will be described. In the above-described first embodiment, a case where the distal end surface 14 includes the first surface 20, the second surface 23 that protrudes from the first surface 20 in the insertion direction ID, and the third surface 25 that protrudes from the first surface 20 in the insertion direction ID has been described. In the second embodiment, a structure of the distal end surface is different. Hereinafter, description of the points common to the first embodiment will not be repeated, and points different from the first embodiment will be described.

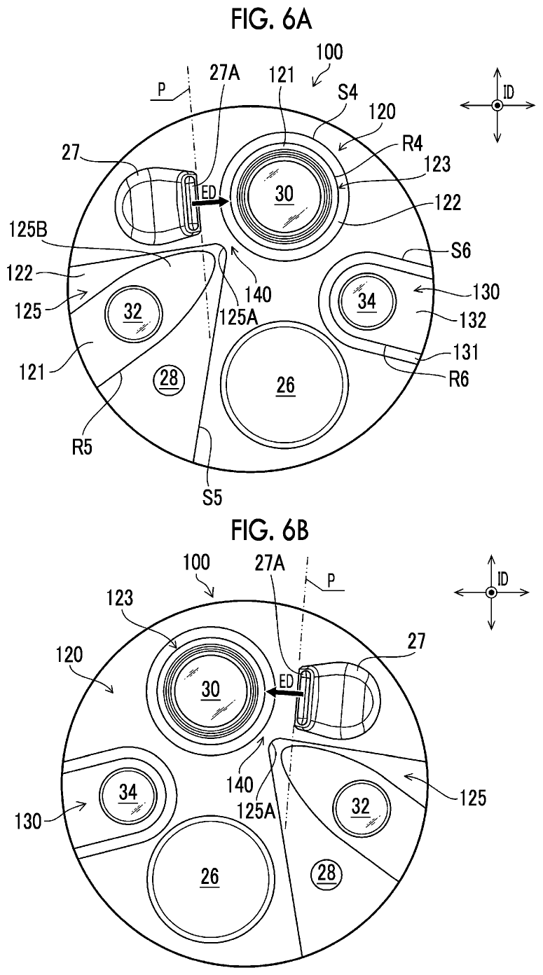

As shown in FIGS. 6A and 6B, a distal end surface 100 includes a first surface 120, a second surface 123 that protrudes from the first surface 120 in the insertion direction ID, a third surface 125 that protrudes from the first surface 120 in the insertion direction ID, and a fourth surface 130 that protrudes from the first surface 120 in the insertion direction ID. In addition, the first surface 120 is provided with the forceps port 26 and the nozzle 27. FIG. 6B is a structure in which the shape of the distal end surface 100 of FIG. 6A is reversed, and the second embodiment may be any case of FIG. 6A or FIG. 6B.

As shown in FIGS. 6A and 6B, as viewed from the insertion direction ID, the second surface 123 has a circular shape, and a ridge line R4 and a tail S4 of the second surface 123 are also formed in a circular shape. The second surface 123 includes a flat surface 121 formed perpendicular to the insertion direction ID, and a stepped portion 122 which is a portion connecting the flat surface 121 and the first surface 120. The observation window 30 is disposed on the flat surface 121.

The fourth surface 130 is disposed between the forceps port 26 and the second surface 123. The fourth surface 130 includes a flat surface 132 formed perpendicular to the insertion direction ID, and a stepped portion 131 which is a portion connecting the flat surface 132 and the first surface 120. The second illumination window 34 is disposed on the flat surface 132.

A ridge line R6 of the fourth surface 130 includes two straight portions extending parallel from an outer periphery of the distal end surface 100 toward a region between the forceps port 26 and the second surface 123, and a curved portion connecting the two straight portions. The flat surface 132 has a bullet-like shape as a whole. The curved portion has a shape following the shape of the second illumination window 34.

Further, a tail S6 of the fourth surface 130 includes two straight portions extending parallel from the outer periphery of the distal end surface 100 toward a region between the forceps port 26 and the second surface 123, and a curved portion connecting the two straight portions. The stepped portion 131 has a bullet-like shape as a whole. The stepped portion 131 of the fourth surface 130 has an inclined surface that is inclined toward the flat surface 132.

The third surface 125 is disposed between the forceps port 26 and the nozzle 27. The third surface 125 comprises a flat surface 121 formed perpendicular to the insertion direction ID, and a stepped portion 122 which is a portion connecting the flat surface 121 and the first surface 120. The first illumination window 32 is disposed on the flat surface 121, and the front water jetting nozzle 28 is disposed on the stepped portion 122.

A ridge line R5 of the third surface 125 includes two straight portions extending parallel in a direction of the second surface 123 from the outer periphery of the distal end surface 100, and a curved portion connecting the two straight portions. The flat surface 121 has a bullet-like shape as a whole.

Further, a tail S5 of the third surface 125 includes two straight portions extending from the outer periphery of the distal end surface 100 in a direction of the second surface 123 and approaching each other. The stepped portion 122 has a substantially triangular shape as a whole. The stepped portion 122 of the third surface 125 has an inclined surface that is inclined toward the flat surface 121. An inclined angle of the stepped portion 122 of the third surface 125 is smaller than an inclined angle of the stepped portion 22 of the third surface 25 of the first embodiment.

An end portion 125A of the third surface 125 is composed of a connection portion between two straight portions of the tail S5 of the third surface 125, and the end portion 125A is located further in the jetting direction ED than the formation position P of the jetting port 27A is. In the second embodiment, the stepped portion 122 constitutes a protruding region 125B. The third surface 125 tapers toward the end portion 125A in the jetting direction ED as a whole.

In the second embodiment shown in FIGS. 6A and 6B, as in the first embodiment, the end portion 125A of the third surface 125 in the jetting direction ED is located further in the jetting direction ED than the formation position of the nozzle 27 is so that the adjacent region AR (particularly, the nozzle side region) (both not shown) on the first surface 120 can be made relatively small. As a result, since it is possible to prevent the cleaning water from remaining on the first surface 120, the movement of the cleaning water to the observation window 30 can be suppressed. As a result, in a case where the air supply operation of the nozzle 27 is performed, the stringing phenomenon of the cleaning water is unlikely to occur, the reflection of the cleaning water on the observation image can be suppressed, and the visibility of the observation window 30 can be improved.

Further, a narrow portion 140 is formed between the second surface 123 and the third surface 125. Due to the narrow portion 140, the negative pressure generated in the forceps port 26 can be applied to the adherent substance remaining in the jetting port peripheral region AR1 (particularly, the residual region) (both not shown) on the first surface 120 so that the adherent substance can be sucked by the forceps port 26. Further, even in a case where the cleaning water remains in the jetting port peripheral region AR1 of the first surface 120, it is possible to suck the cleaning water into the forceps port 26 through the suction operation in the same manner as the adherent substance.

### MODIFICATION EXAMPLES

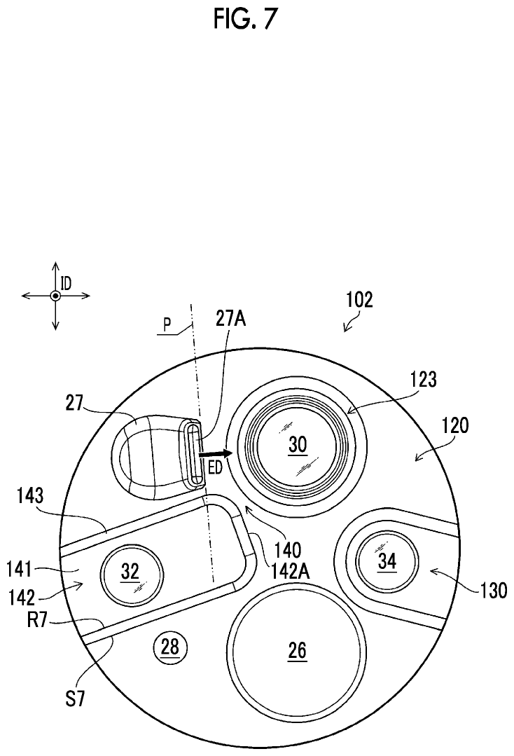

Hereinafter, modification examples of the distal end surface of the second embodiment will be described. In FIGS. 7 to 9A and 9B, the same parts as those in the above-described second embodiment are designated by the same references, and the description thereof will not be repeated.

### Modification Example 1

FIG. 7 is a view for explaining Modification Example 1. In a distal end surface 102 shown in FIG. 7, as viewed from the insertion direction ID, a shape of a third surface 142 is different from the shape of the third surface 125 of the above-described second embodiment, and the third surface 142 in Modification Example 1 has a substantially rectangular shape as a whole.

The third surface 142 includes a flat surface 141 formed perpendicular to the insertion direction ID, and a stepped portion 143 which is a portion connecting the flat surface 141 and the first surface 120. The first illumination window 32 is disposed on the flat surface 141. An inclined angle of the stepped portion 143 is larger than an inclined angle of the stepped portion 122 of the second embodiment.

A ridge line R7 between the flat surface 141 and the stepped portion 143 of the third surface 142 includes three straight portions and is composed of two parallel straight portions extending from an outer periphery of the distal end surface 102 toward the jetting direction ED and one straight portion that connects the two straight portions. In addition, a tail S7 between the first surface 120 and the stepped portion 143 includes three straight portions as in the ridge line R7 and is composed of two parallel straight portions extending from the outer periphery of the distal end surface 102 toward the jetting direction ED and one straight portion that connects the two straight portions. The one straight portion that connects the two straight portions constitutes an end portion 142A, and the end portion 142A is located further in the jetting direction ED than the formation position P of the jetting port 27A is.

In Modification Example 1, even in a case where a space between the forceps port 26 and the nozzle 27 is wider than that of the second embodiment, by making the third surface 142 in a substantially rectangular shape, the adjacent region AR on the first surface 120 can be made small. As a result, since the cleaning water can be prevented from remaining on the first surface 120, the movement of the cleaning water to the observation window 30 can be suppressed.

Further, since an area of the flat surface 141 is larger than that of the flat surface 121 of the second embodiment, a degree of freedom in the disposition of the first illumination window 32 is increased.

### Modification Example 2

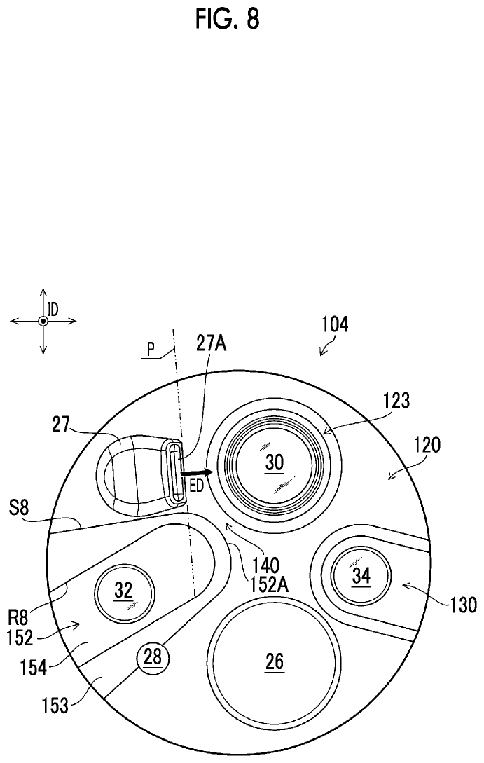

FIG. 8 is a view for explaining Modification Example 2. In a distal end surface 104 shown in FIG. 8, as viewed from the insertion direction ID, a shape of a third surface 152 is different from the shape of the third surface 125 of the above-described second embodiment, and the third surface 152 in Modification Example 2 has a substantially bullet-like shape as a whole.

The third surface 152 includes a flat surface 154 formed perpendicular to the insertion direction ID, and a stepped portion 153 which is a portion connecting the flat surface 154 and the first surface 120. The first illumination window 32 is disposed on the flat surface 154. An inclined angle of the stepped portion 153 is larger than the inclined angle of the stepped portion 122 of the second embodiment and smaller than the inclined angle of the stepped portion 143 of Modification Example 1.

A ridge line R8 between the flat surface 154 and the stepped portion 153 of the third surface 152 is composed of two straight portions extending parallel from an outer periphery of the distal end surface 104 toward the jetting direction ED and one curved portion connecting the two straight portions.

In addition, in the same manner as the ridge line R8, a tail S8 between the first surface 120 and the stepped portion 153 is composed of two straight portions extending from the outer periphery of the distal end surface 104 toward the jetting direction ED and approaching each other, and one curved portion that connects the two straight portions.

The curved portion of the tail S8 constitutes an end portion 152A, and the end portion 152A is located further in the jetting direction ED than the formation position P of the jetting port 27A is.

In Modification Example 2, the third surface 152 is formed in a substantially bullet-like shape so that even in a case where a space between the forceps port 26 and the nozzle 27 is wider than that of the second embodiment, the third surface 152 can be brought close to the nozzle 27, and the adjacent region AR (particularly, the nozzle side region) (both not shown) on the first surface 120 can be made relatively small. As a result, since the cleaning water can be prevented from remaining on the first surface 120, the movement of the cleaning water to the observation window 30 can be suppressed.

Further, since an area of the flat surface 154 is larger than that of the flat surface 121 of the second embodiment, a degree of freedom in the disposition of the first illumination window 32 is increased.

### Modification Example 3

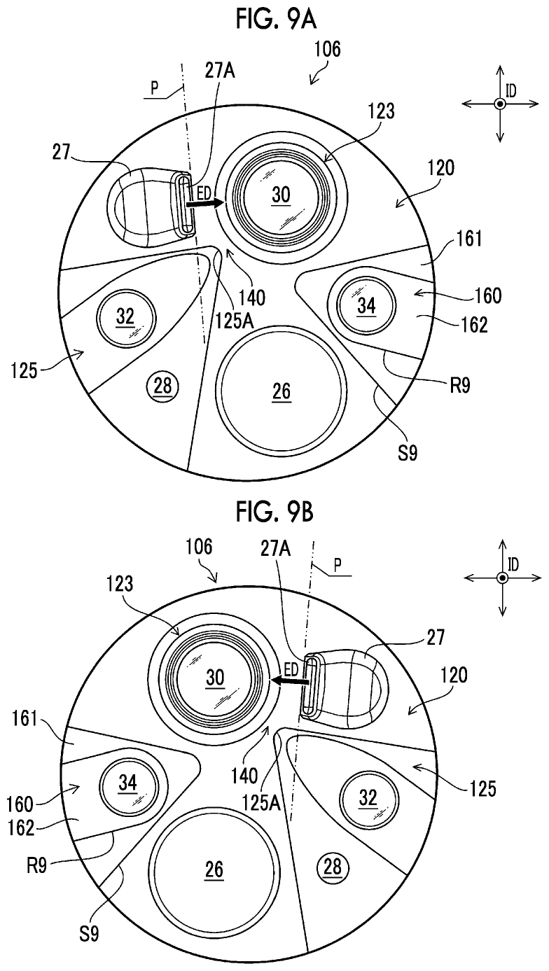

FIGS. 9A and 9B are views for explaining Modification Example 3. In a distal end surface 106 shown in FIGS. 9A and 9B, as viewed from the insertion direction ID, a shape of a fourth surface 160 is different from the shape of the fourth surface 130 of the above-described second embodiment, and in Modification Example 3, the fourth surface 160 has a substantially triangular shape as a whole. FIG. 9B shows a structure in which the shape of the distal end surface 106 of FIG. 9A is reversed, and Modification Example 3 may be any case of FIG. 9A or FIG. 9B.

As shown in FIGS. 9A and 9B, the fourth surface 160 is disposed between the forceps port 26 and the second surface 123. The fourth surface 160 includes a flat surface 162 formed perpendicular to the insertion direction ID, and a stepped portion 161 which is a portion connecting the flat surface 162 and the first surface 120. The second illumination window 34 is disposed on the flat surface 162.

A ridge line R9 of the fourth surface 160 is composed of two straight portions extending parallel from an outer periphery of the distal end surface 106 toward a region between the forceps port 26 and the second surface 123, and a curved portion connecting the two straight portions. The flat surface 162 has a substantially bullet-like shape as a whole. The curved portion has a shape following the shape of the second illumination window 34.

Further, a tail S9 of the fourth surface 160 includes two straight portions that approach each other from the outer periphery of the distal end surface 106 toward a region between the forceps port 26 and the second surface 123. The stepped portion 161 has a substantially triangular shape as a whole. The stepped portion 161 of the fourth surface 160 has an inclined surface that is inclined toward the flat surface 162.

Modification Example 3 comprises the third surface 125 as in the second embodiment. Therefore, as in the second embodiment, by making the adjacent region AR (particularly, the nozzle side region) (both not shown) on the first surface 120 relatively small, the movement of the cleaning water to the observation window 30 can be suppressed. Further, due to the narrow portion 140, the negative pressure generated in the forceps port 26 can be applied to the adherent substance remaining in the jetting port peripheral region AR1 (particularly, the residual region) (both not shown) on the first surface 120 so that the adherent substance can be sucked from the forceps port 26. Further, even in a case where the cleaning water remains in the jetting port peripheral region AR1 of the first surface 120, it is possible to suck the cleaning water into the forceps port 26 through the suction operation in the same manner as the adherent substance.

### DISCLOSURE OF OTHER EMBODIMENTS OF THE INVENTION

Next, other embodiments 1 and 2 of the invention (hereinafter, referred to as Other Embodiments 1 and 2) will be described.

### Other Embodiment 1

As shown in FIG. 1, the distal end portion 7 is connected to the bendable portion 6, and the distal end portion 7 can be directed in a desired direction by operating the angle knobs 8 and 9.

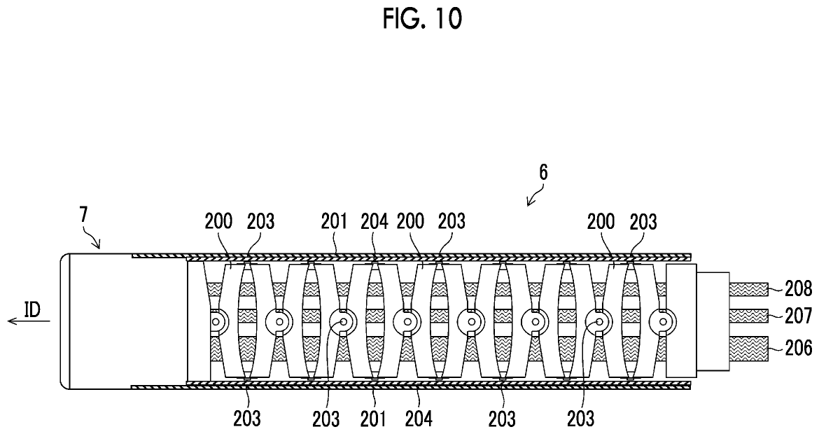

FIG. 10 is a view showing a schematic configuration of the distal end portion 7 and the bendable portion 6. As shown in FIG. 10, the bendable portion 6 comprises a plurality of metal rings 200 (also referred to as nodal rings or bridges) connected along the insertion direction ID, a net 204 consisting of a metal wire braid covering an outer periphery of each ring 200, and a tube 201 covering the net 204. The rings 200 adjacent to each other are rotatably connected to each other by a caulking pin 203. The ring 200 and the caulking pin 203 are made of metal.

A plurality of angle wires (not shown) are inserted into the respective rings 200. One end side of each angle wire is connected to the distal end portion 7, and the other end side thereof is connected to a rotating member (not shown) that is rotationally operated by the angle knobs 8 and 9. Thus, the bendable portion 6 is remotely operated to be bent (angle operation) by rotating the angle knobs 8 and 9 provided in the operating part 3.

For example, a forceps tube 206 communicating with the forceps port 26 of the distal end portion 7, an air/water supply tube 207 communicating with the nozzle 27, a water jet (WJ) tube 208 communicating with the front water jetting nozzle 28, and the like, are inserted into the bendable portion 6.

As the air/water supply tube 207 and the WJ tube 208 are generally required to have flexibility against bending, airtightness, and chemical resistance, a fluororesin tube, for example, a polytetrafluoroethylene (PTFE) tube is often used.

However, in a case where the PTFE tube used for the air/water supply tube 207 and the WJ tube 208 comes into contact with the caulking pin 203 in the bendable portion 6, a leakage of airtightness due to perforation of the PTFE tube is caused. Therefore, it is necessary to improve a strength of the PTFE tube with respect to the caulking pin 203, for example, by covering the PTFE tube with a close contact spring made of steel special use stainless (SUS).

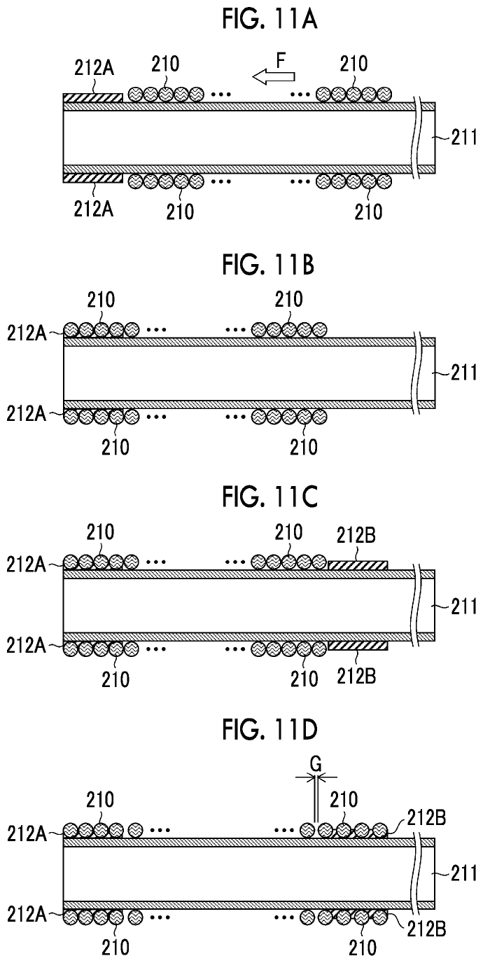

FIGS. 11A to 11D are views for explaining a first method of covering a PTFE tube 211 with a close contact spring 210. As shown in FIG. 11A, an adhesive 212A is applied to one end of the PTFE tube 211. The close contact spring 210 externally mounted on the PTFE tube 211 is moved in a moving direction F (direction of the adhesive 212A) indicated by an arrow.

Next, as shown in FIG. 11B, the close contact spring 210 is moved to a position of the adhesive 212A, and one end of the close contact spring 210 is bonded to the PTFE tube 211 via the adhesive 212A.

Next, as shown in FIG. 11C, an adhesive 212B is applied to a position away from the other end of the close contact spring 210 on the PTFE tube 211. This is because, since the close contact spring 210 is wound without a gap, the adhesive 212B cannot be applied between the close contact spring 210 and the PTFE tube 211.

Finally, as shown in FIG. 11D, the other end of the close contact spring 210 is stretched to a position of the adhesive 212B, and the close contact spring 210 and the PTFE tube 211 are bonded to each other via the adhesive 212B.

In such a first method, since it is necessary to stretch and bond the close contact spring 210, the close contact spring 210 is not completely brought into close contact, and a gap G is generated in the close contact spring 210. The caulking pin 203 enters the gap G, and the caulking pin 203 causes perforation in the PTFE tube 211.

Therefore, in Other Embodiment 1, by using a close contact spring having a sparsely wound portion at least at an end portion, an occurrence of perforation in the PTFE tube 211 by the caulking pin 203 is suppressed.

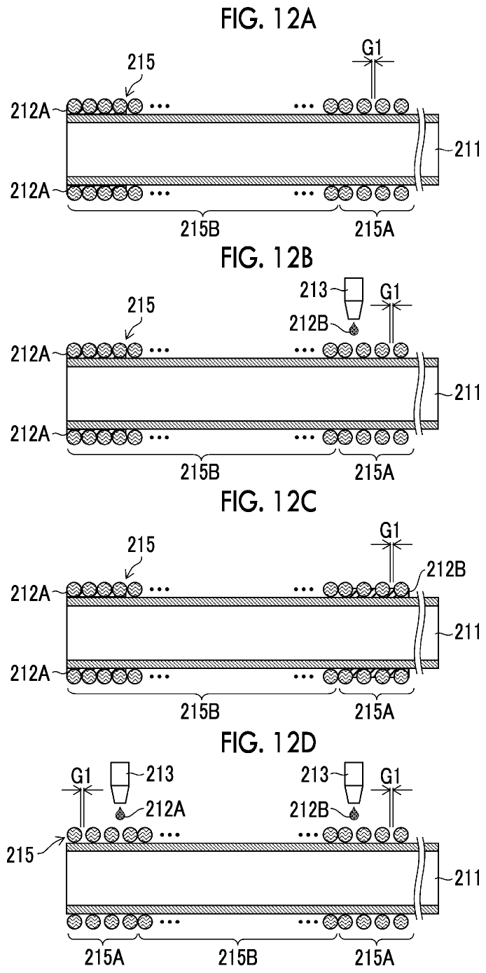

FIGS. 12A to 12D are views for explaining a second method of covering the PTFE tube 211 with a close contact spring 215 comprising a sparsely wound portion 215A at an end portion.

As shown in FIG. 12A, the adhesive 212A is applied to one end of the PTFE tube 211, and the close contact spring 215 is moved to the adhesive 212A side. The close contact spring 215 comprises the sparsely wound portion 215A at the end portion, and the sparsely wound portion 215A is wound with a predetermined gap G1 in advance. Meanwhile, the close contact spring 215 has a close contact portion 215B that is closely wound, except for at the sparsely wound portion 215A. One end (close contact portion 215B) of the close contact spring 215 is bonded to the PTFE tube 211 via the adhesive 212A.

Next, as shown in FIG. 12B, an applicator 213 applies the adhesive 212B from above the sparsely wound portion 215A. Since the predetermined gap G1 is provided in advance in the sparsely wound portion 215A, the adhesive 212B reaches the PTFE tube 211.

Finally, as shown in FIG. 12C, the close contact spring 215 and the PTFE tube 211 are bonded to each other via the adhesive 212B without stretching the close contact spring 215.

Since the close contact spring 215 of Other Embodiment 1 is composed of the close contact portion 215B where the spring is in close contact at a portion other than the sparsely wound portion 215A, the caulking pin 203 can be prevented from coming into contact with the PTFE tube 211. Since the gap G1 between the sparsely wound portion 215A is filled with the adhesive 212B, it is possible to prevent the caulking pin 203 from coming into contact with the PTFE tube 211. By using the close contact spring 215 comprising the sparsely wound portion 215A at the end portion, it is possible to improve a strength of the PTFE tube 211 with respect to the caulking pin 203 in an entire range of the close contact spring 215.

In FIGS. 12A to 12C, the close contact spring 215 including the sparsely wound portion 215A at the end portion is illustrated, but the present invention is not limited to this configuration. The close contact spring 215 may be configured to include the sparsely wound portions 215A at both end portions, and the close contact portion 215B provided between the two sparsely wound portions 215A. As shown in FIG. 12D, both end portions are provided with the sparsely wound portions 215A. Accordingly, after the close contact spring 215 is externally mounted on the PTFE tube 211, the adhesives 212A and 212B can be applied from above the sparsely wound portions 215A at both end portions using the applicator 213. In the close contact spring 215 shown in FIG. 12D, the strength of the PTFE tube 211 with respect to the caulking pin 203 can be improved in the entire range. Further, it is possible to easily determine a relative positional relationship between the PTFE tube 211 and the close contact spring 215.

### Other Embodiment 2

As shown in FIG. 1, the endoscope 1 comprises the treatment tool inlet port 12 in the operating part 3. A treatment tool is inserted from the treatment tool inlet port 12 and is led out from the forceps port 26 of the distal end portion 7. An operator uses the treatment tool to perform a treatment such as incision or excision of biological tissue, or coagulation of the biological tissue to stop bleeding.

As an example of the treatment using the endoscope 1, endoscopic submucosal dissection (ESD) is known. ESD is a treatment in which a lesioned mucous membrane is excised using the treatment tool in a case where a lesion part such as a tumor is found at a mucous membrane part of a body cavity inner wall of an esophagus, a stomach, a duodenum, a large intestine, and the like during an endoscopy.

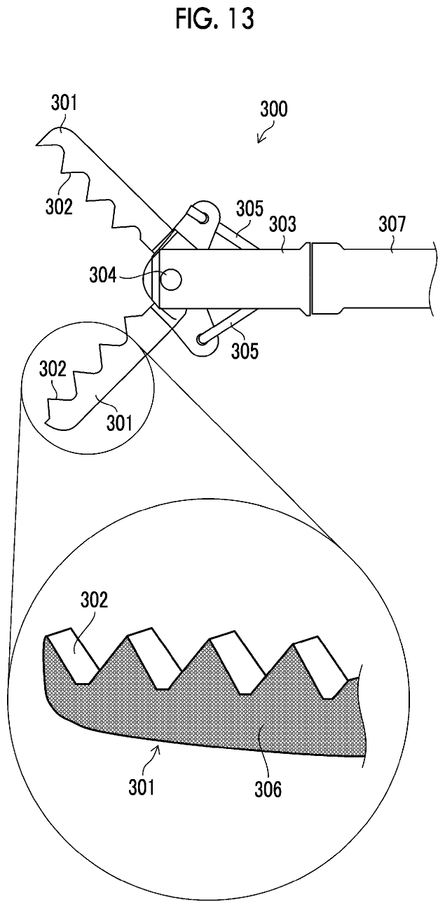

As the treatment tool used for ESD, a high-frequency treatment tool is known. FIG. 13 shows an example of a structure (treatment tool main body) of a distal end portion of the high-frequency treatment tool. As shown in FIG. 13, a treatment tool main body 300 comprises a pair of claw portions 301 and 301. The pair of claw portions 301 comprises a plurality of tapered blades 302 on opposing sides. The pair of claw portions 301 is supported by a shaft 304 of a holding member 303. A connection wire 305 is connected to each of end portions of the pair of claw portions 301. The claw portion 301 and the connection wire 305 are electrically connected to each other.

The holding member 303 is connected to a flexible sheath 307. An operation wire (not shown) connected to the connection wire 305 is inserted through the flexible sheath 307, and the pair of claw portions 301 can be opened and closed by pushing and pulling the operation wire.

Since a high-frequency wave is applied to the treatment tool main body 300, the claw portion 301 is covered with an insulating member 306, except for at the blade 302 portion, and a portion other than a connecting portion between the claw portion 301 and the connection wire 305 is also covered with an insulating member or the like, as shown in an enlarged view of FIG. 13.

In a case where a metal portion is provided in the forceps port 26, sparks may occur in the metal portion due to interposition of a body fluid or the like in a case where the treatment tool main body 300 is led out from the forceps port 26 of the distal end portion 7 and the high-frequency treatment tool is energized, which causes a resin portion of the distal end portion 7 to be damaged. Therefore, it is desirable to provide the distal end portion 7 with a structure that prevents sparks.

Therefore, in Other Embodiment 2, a part in the forceps port 26 of the distal end portion 7 is resinified to prevent generation of a spark between the treatment tool main body 300 and the metal portion in a case where the high-frequency treatment tool is energized.

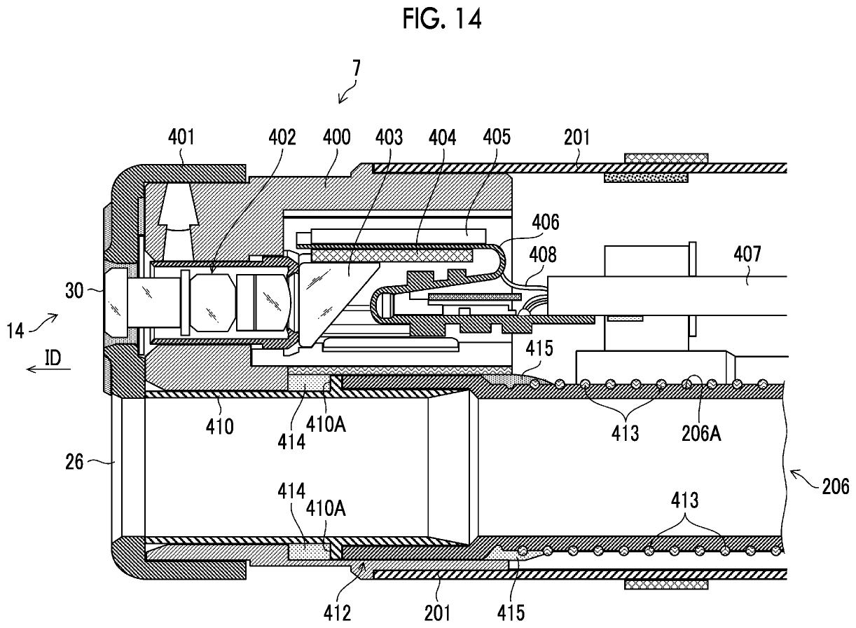

FIG. 14 shows a cross-sectional view of the distal end portion 7. As shown in FIG. 14, the distal end portion 7 comprises a distal end portion body 400 made of a metal and an annular distal end cap 401 made of a resin, which is attached to a distal end side of the distal end portion body 400. The observation window 30 is disposed on the distal end surface 14 of the distal end portion 7. A lens group 402, a prism 403, an imaging element 404, a main board 405, a sub-board 406, and a signal cable 407 are disposed on a rear side (proximal end side) of the observation window 30. The imaging element 404 is attached to the main board 405. A component or the like that cannot be attached to the main board 405 is attached to the sub-board 406. A multi-core cable is used as the signal cable 407. The signal cable 407 includes a plurality of strands 408, and the plurality of strands 408 are electrically connected to the main board 405 and the sub-board 406.

In the distal end portion body 400, a forceps pipe 410 made of a resin, which communicates with the forceps port 26, is disposed on a rear side (proximal end side) of the forceps port 26. The forceps pipe 410 is made of, for example, polysulfone. The forceps pipe 410 has a tubular shape having openings on a distal end side and a proximal end side. A flange 410A is provided on an outer periphery of the forceps pipe 410 along a circumferential direction. The forceps tube 206 made of a resin is connected to a proximal end side of the forceps pipe 410. The forceps tube 206 is composed of, for example, a PTFE tube. A connection portion (a portion where the forceps pipe 410 is covered with the forceps tube 206) 412 between the forceps pipe 410 and the forceps tube 206 is disposed inside the distal end portion body 400. That is, the forceps pipe 410 does not protrude to the proximal end side with respect to the distal end portion body 400.

A spiral groove 206A is formed on an outer periphery of the forceps tube 206 in a circumferential direction. A metal wire 413 is wound in a spiral shape along the groove 206A. With the metal wire 413, it is possible to improve kink resistance of the forceps tube 206.

A space is formed by the flange 410A and the distal end portion body 400, and an adhesive 414 is provided in this space along the flange 410A. Further, an adhesive 415 is provided between the distal end portion body 400 and the forceps tube 206 on the proximal end side of the distal end portion body 400.

Next, features of the distal end portion 7 of Other Embodiment 2 will be described.

Since the forceps pipe 410 is made of a resin in the distal end portion 7, an entire path (the forceps tube 206, the forceps pipe 410, and the distal end cap 401) through which the treatment tool main body 300 of the high-frequency treatment tool passes is made of a resin. That is, since the path of the treatment tool main body 300 does not include a metal portion in the distal end portion 7, the generation of a spark can be prevented.

In addition, since the connection portion 412 is disposed inside the distal end portion body 400, it is possible to suppress breakage of the forceps pipe 410 made of a resin.

In addition, since the forceps pipe 410 comprises the flange 410A, a distal end side of the forceps tube 206 comes into contact with the flange 410A in a case where the forceps tube 206 is externally mounted on the forceps pipe 410. As a result, it is possible to easily position the forceps pipe 410 and the forceps tube 206.

In addition, it is possible to secure a region for storing the adhesive 414 by forming a space with the flange 410A and the distal end portion body 400. In addition, by providing the adhesive 415, it is possible to prevent the forceps tube 206 from swinging and coming into contact with the distal end portion body 400 at this location, and to suppress damage of the forceps tube 206.

The endoscope according to the embodiments of the present invention has been described above in detail, but the present invention may include some improvements or modifications without departing from the scope of the present invention.

### EXPLANATION OF REFERENCES

1: endoscope
2: insertion part
3: operating part
4: universal cord
5: soft portion
6: bendable portion
7: distal end portion
8, 9: angle knob
10: air/water supply button
11: suction button
12: treatment tool inlet port
14: distal end surface
20: first surface
20A: forceps port disposition surface
20B: nozzle disposition surface
21: flat surface
22: stepped portion
23: second surface
24: stepped portion
25: third surface
25A: end portion
25B: protruding region
26: forceps port
27: nozzle
27A: jetting port
28: front water jetting nozzle
29: flat surface
30: observation window
32: first illumination window
34: second illumination window
40: narrow portion
50: distal end surface
52: third surface
52A: end portion
53: stepped portion
54: flat surface
100, 102, 104, 106: distal end surface
120: first surface
121: flat surface
122: stepped portion
123: second surface
125: third surface
125A: end portion
125B: protruding region
130: fourth surface
131: stepped portion
132: flat surface
140: narrow portion
141: flat surface
142: third surface
142A: end portion
143: stepped portion
152: third surface
152A: end portion
153: stepped portion
154: flat surface
160: fourth surface
161: stepped portion
162: flat surface
200: ring
201: tube
203: caulking pin
204: net
206: forceps tube
206A: groove
207: air/water supply tube
208: WJ tube
210: close contact spring
211: PTFE tube
212A: adhesive
212B: adhesive
215: close contact spring
215A: sparsely wound portion
215B: close contact portion
300: treatment tool main body
301: claw portion
302: blade
303: holding member
304: shaft
305: connection wire
306: insulating member
307: flexible sheath
400: distal end portion body
401: distal end cap
402: lens group
403: prism
404: imaging element
405: main board
406: sub-board
407: signal cable
408: strand
410: forceps pipe
410A: flange
412: connection portion
413: metal wire
414, 415: adhesive
AR: adjacent region
AR1: jetting port peripheral region
ED: jetting direction
G, G1: gap
ID: insertion direction
L1, L2: distance
P: formation position
R1, R2, R3, R4, R5, R6, R7, R8, R9: ridge line
S1, S2, S3, S4, S5, S6, S7, S8, S9: tail

What is claimed is:

1. An endoscope comprising:
an insertion part that is inserted into a subject;
a first surface that is disposed on a distal end surface of the insertion part;
a forceps port that is disposed on the first surface;
a second surface that protrudes from the first surface along an insertion direction of the insertion part;
an observation window that is disposed on the second surface;
a nozzle that is disposed on the first surface and that jets a fluid from a jetting port toward the observation window;
a third surface that protrudes from the first surface along the insertion direction of the insertion part; and
a first illumination window that is disposed on the third surface,
wherein the third surface is located between the nozzle and the forceps port, and an end portion of the third surface in a jetting direction of the fluid is located further in the jetting direction than a formation position of the jetting port is.

2. The endoscope according to claim 1,
wherein the third surface comprises a protruding region including the end portion and extending in the jetting direction between a formation position of the first illumination window and the nozzle.

3. The endoscope according to claim 1,
wherein the third surface is tapered toward the end portion in the jetting direction along the nozzle from an outer periphery of the distal end surface.

4. The endoscope according to claim 1,
wherein a ridge line of the third surface on a side of the nozzle extends along the nozzle.

5. The endoscope according to claim 1,
wherein a second illumination window is disposed on the second surface on a side opposite to the nozzle with the observation window interposed therebetween.

6. The endoscope according to claim 1, further comprising:
a narrow portion formed by the second surface and the third surface.

7. The endoscope according to claim 6,
wherein a distance between a tail of the second surface and a tail of the third surface in the narrow portion is shorter than a distance between the jetting port and the tail of the second surface.
